(19) United States
(12) Patent Application Publication
(10) Pub. No.: US 2025/0093307 A1
(43) Pub. Date: Mar. 20, 2025

(54) PHOTOACOUSTIC DETECTING DEVICE COMPRISING A MEMBRANE FORMING A CONTACT FACE

(71) Applicants: COMMISSARIAT A L'ENERGIE ATOMIQUE ET AUX ENERGIES ALTERNATIVES, Paris (FR); ECLYPIA, Grenoble (FR)

(72) Inventors: Kévin JOURDE, Grenoble (FR); Jean-Guillaume COUTARD, Saint Pancrasse (FR); Etienne NIORTHE, Grenoble Cedex 09 (FR); Thibault LE ROUX MALLOUF, Grenoble (FR)

(73) Assignees: COMMISSARIAT A L'ENERGIE ATOMIQUE ET AUX ENERGIES ALTERNATIVES, Paris (FR); ECLYPIA, Grenoble (FR)

(21) Appl. No.: 18/723,949

(22) PCT Filed: Dec. 24, 2022
(86) PCT No.: PCT/EP2022/087836

§ 371 Date: Jun. 25, 2024

(30) Foreign Application Priority Data
Dec. 26, 2021 (FR) FR2114530

(51) Int. Cl. G01N 29/24; G01N 29/02

(52) U.S. Cl. CPC G01N 29/2418; G01N 29/02; G01N 2291/022

(57) ABSTRACT

A photoacoustic detecting device to be applied, via a contact face, against a medium to be analyzed, the device comprising: a hollow cavity; a light source that is pulsed or amplitude-modulated; an acoustic detector configured to detect an acoustic wave extending through the cavity, the device further comprises an interface membrane, forming the contact face, the interface membrane being configured to: form an interface between the gas, filling the cavity, and the medium to be analyzed; block passage of a liquid or gel between the medium to be analyzed and the cavity; and generate an acoustic pressure wave inside the cavity, under the effect of a variation in the temperature of the interface membrane, the temperature variation of the interface membrane being induced by heating of the medium resulting from illumination of the medium.

### TECHNICAL FIELD

The technical field of the invention is detection of an analyte via photoacoustic detection.

### PRIOR ART

Photoacoustic detection is based on detection of an acoustic wave generated under the effect of absorption, by an analyzed medium, of an incident electromagnetic wave that is pulsed or amplitude-modulated. The acoustic wave is formed following heating, under the effect of absorption of the incident wave, of molecules of interest present in the analyzed medium. The heating leads to a modulated thermal expansion of the medium, said expansion being the origin of the acoustic wave.

Photoacoustic detection may be made specific to one particular analyte, by adjusting the wavelength of the incident electromagnetic wave to an absorption wavelength of the analyte. Photoacoustic detection has thus been applied to the detection of gas species in a gas, or to the detection of the presence of particular molecules in biological tissues. The wavelength of the incident wave is frequently located in the infrared.

A photoacoustic detecting device comprises an amplitude-modulated light source, a laser source for example, that is activated at a frequency comprised between several tens of Hz and several tens of kHz. The modulation frequency defines the frequency of the photoacoustic wave resulting from the periodic heating of the molecules of interest present in the medium to be analyzed. A photoacoustic detecting device comprises an acoustic detector configured to detect the periodic photoacoustic wave. A response function of the photoacoustic device may be calibrated, so as to establish a correlation between the measured amplitude of the pressure oscillations and the amount of analyte in the analyzed medium.

Devices have been described that allow photoacoustic detection to be applied to biological media, for example to quantify certain biomolecules such as glucose. Examples of devices are described in US2014/0073899, or in US20210302387. In these devices, the analyzed medium is heated periodically under the effect of a periodic illumination. The periodic heating of the medium propagates to an interface between the medium and an air-filled cavity. The temperature variation at the interface results in generation of a periodic pressure wave in the cavity.

The inventors have designed a photoacoustic detecting device based on the same principle as the devices mentioned in the preceding paragraph, but suitable for being applied to a liquid medium.

### SUMMARY OF THE INVENTION

A first subject of the invention is a photoacoustic detecting device, intended to be applied, via a contact face, against a medium to be analyzed, the device comprising:
a hollow cavity that is filled with a gas, and that opens onto the contact face;
a light source that is configured to emit, when it is activated, an incident light beam in an emission spectral band, through the cavity, to the contact face, the incident light beam being pulsed or amplitude-modulated;
an acoustic detector connected to the cavity, and configured to detect an acoustic wave propagating through the cavity;
   such that, under the effect of illumination of the medium by the incident light beam, the acoustic detector detects an acoustic wave produced by heating of the medium;
   the device being characterized in that it comprises an interface membrane, forming the contact face, the interface membrane being configured to:
form an interface between the gas, filling the cavity, and the medium to be analyzed;
block passage of the medium to be analyzed into the cavity.

According to one embodiment, the interface membrane is unapertured.

According to one embodiment, the interface membrane comprises through-apertures of radius less than 50 μm or than 30 μm. The membrane may comprise a hydrophobic coating in the through-apertures.

According to one embodiment:
the light source is arranged such that, when it is activated, the incident light beam passes through the interface membrane before reaching the medium to be analyzed;
the interface membrane comprises an intersecting segment corresponding to a portion of the membrane passed through by the light beam;
at least in the intersecting segment, the interface membrane is made of a transmissive material having a transmittance higher than 0.4 in the emission spectral band.

The interface membrane then allows the amount of light propagating through the medium to be optimized. The transmissive material may be at least one material selected from: Si, Ge, AlN, ZnSe, BaF₂, CaF₂, KBr, ZnS, sapphire. The membrane may be monolithic and made of the transmissive material.

According to one embodiment, the interface membrane is removable.

Advantageously,
the interface membrane extends between an internal surface, making contact with the gas filling the cavity, and an external surface, intended to be applied against the medium to be analyzed;
the internal surface of the interface membrane comprises an anti-reflection coating or micro-structuring configured to minimize reflection of the light beam;
and/or the external surface of the interface membrane comprises an anti-reflection coating or micro-structuring configured to minimize reflection of the light beam.

The thickness of the interface membrane may be comprised between 20 μm and 1 mm.

According to one embodiment:
the cavity is bounded by a distal membrane, the distal membrane lying opposite the interface membrane, so that the cavity extends between the distal membrane and the interface membrane;
the light source is arranged in such a way that, when it is activated, the incident light beam passes through the distal membrane before reaching the interface membrane.

The distal membrane may comprise or be made of a material having a transmittance higher than 0.4 in the emission spectral band. It may in particular be a question of a material chosen from Si, Ge, AlN, ZnSe, BaF₂, CaF₂, KBr, ZnS, sapphire.

The distal membrane and the transmissive membrane may be made of the same material.

According to one embodiment:
the cavity is bounded by a distal wall and a lateral wall, the lateral wall extending between the distal wall and the interface membrane;
the membrane extends between opposite edges of the sidewall.

The volume of the cavity may be less than 50 μL.

A second subject of the invention is a method for detecting an analyte in a medium, the analyte absorbing light at an absorption wavelength, the method comprising:
applying a device according to the first subject of the invention against the medium, so that the interface membrane makes contact with the medium;
activating the light source, the emission spectral band containing the absorption wavelength of the analyte;
detecting a photoacoustic pressure wave by means of the acoustic detector and estimating an amount of analyte depending on the detected photoacoustic pressure wave, and more precisely depending on an amplitude of the photoacoustic pressure wave.

The medium may be or comprise a liquid or be a gel.

The light source may be pulsed or amplitude-modulated, with a pulse frequency or modulation frequency less than 500 Hz.

The invention will be better understood on reading the description of the examples of embodiment that are presented, in the rest of the description, with reference to the figures listed below.

### FIGURES

FIG. 1 shows one example of a photoacoustic detecting device.

FIG. 2 shows one example of an interface membrane.

FIGS. 3A and 3B show one example of application of the device against a duct through which a liquid flows.

FIG. 4 shows another example of an interface membrane.

FIG. 5 shows modeling of the amplitude of thermal oscillations generated in the medium and cavity during periodic illumination of the medium (y-axis), along a transverse axis perpendicular to the interface membrane. The x-axis corresponds to the position along the transverse axis.

FIG. 6 shows modeling of an amplitude of thermal oscillations (y-axis) generated in the analyzed medium and in the cavity, under the effect of a periodic illumination of the medium, as a function of the modulation frequency of the illumination beam (x-axis).

FIGS. 7A to 7C schematically show steps of a process for manufacturing the cavity and interface membrane of the device.

### DESCRIPTION OF PARTICULAR EMBODIMENTS

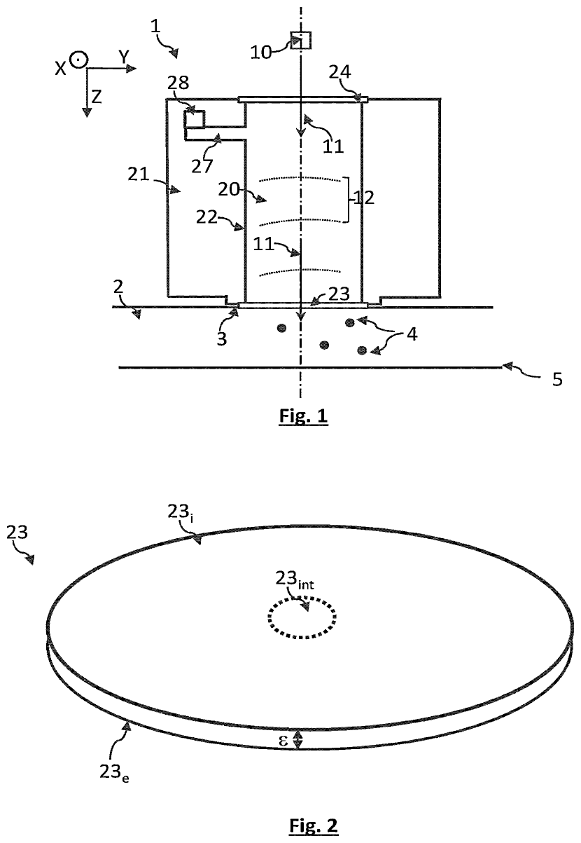

FIG. 1 schematically shows a device 1 allowing the invention to be implemented. The device 1 is configured to be applied against a medium 2 to be analyzed. The device comprises a contact face 3, which is intended to be applied against the medium to be analyzed. In the example shown, the medium 2 is a liquid flowing through a duct 5. The liquid does not need to be flowing. The invention is applicable to analysis of a static liquid placed in a container. The invention is also applicable to analysis of a solid or gel.

The device comprises a light source 10, configured to emit a light beam 11 that propagates to the medium 2 to be analyzed. The light source 10 is pulsed or amplitude-modulated. The light beam 11 is emitted in an emission spectral band Δλ comprising an absorption wavelength λₐ of an analyte 4 present in the medium. One objective of the device 1 is to detect the presence of the analyte 4 and potentially to estimate a concentration thereof.

The analyte 4 may be a molecule present in a fluid, potentially a body fluid or a fluid used in an industrial process. It may for example be a question of glucose, or alcohol, ethanol for example.

The emission spectral band Δλ preferably lies in the visible or in the infrared and for example extends between wavelengths of 2 μm and 15 μm. Preferably, the emission spectral band Δλ is narrow enough for the device 1 to be specific to a single analyte. When the analyte is glucose, the emission spectral band is centered on an absorption wavelength of glucose, for example a wavelength corresponding to a wavenumber of 1034 cm⁻¹. The light source 10 may notably be a pulsed laser source and may for example be a wavelength-tunable quantum cascade laser (QCL). According to other embodiments, the light source may be a filament-type source or a light-emitting diode. According to these embodiments, it is preferable to associate the light source with a bandpass filter in order to define a sufficiently narrow emission spectral band centered on the absorption wavelength in question.

The device 1 is intended to be applied against the medium to be analyzed 2. It comprises a body 21 in which a cavity has been formed. The cavity 20 is filled with a gas, air for example. The cavity 20 opens onto an interface membrane 23. The interface membrane 23 forms an interface between the cavity 20 and the medium to be analyzed 2. The membrane is configured to block passage of liquid or gel between the analyzed medium 2 and the cavity 20. Thus, the membrane is non-porous to a liquid or gel. The membrane may be porous to a gas, as described below. In the example shown, the membrane is flush with the internal surface of the duct through which the liquid flows. The membrane may be flexible or rigid.

The light source 10 is configured to emit the light beam 11 in such a way that the latter reaches the medium to be analyzed 2 at a normal or substantially normal angle of incidence. By substantially normal, what is meant is normal to within an angular tolerance of ±30°. Preferably, the light beam 11 passes through the interface membrane 23 before reaching the medium to be analyzed 2.

Under the effect of the presence of an analyte 4 in the medium 2, an acoustic 20) wave, called the photoacoustic wave 12, is formed. The photoacoustic wave 12 is an acoustic wave that forms as a result of periodic heating of the medium by the light beam 11, the latter being pulsed or amplitude-modulated. The periodic heating is transmitted, by thermal conduction, to the interface between the medium 2 and the cavity 20. At the interface, a periodic photoacoustic wave is formed and propagates through the cavity 20. The photoacoustic wave, and in particular its amplitude, is detected by an acoustic detector 28. The acoustic detector 28 is connected to the cavity 20 by an acoustic channel 27. The acoustic detector may be a microphone, having a detection spectral range comprising the frequency of the photoacoustic wave. The photoacoustic wave is amplitude modulated with the pulse or amplitude-modulation frequency of the light beam 11. Thus, at the acoustic detector, the pressure is amplitude modulated. The amplitude of the measured acoustic wave is correlated with the concentration of the analyte in the medium.

The cavity 20 is bounded by the interface membrane 23. It also extends, in the body 21, between a lateral wall 22 and a distal wall 24. The lateral wall 22 extends around an axis parallel to a transverse axis Z, between the interface membrane 23 and the distal wall 24. The transverse axis Z is perpendicular to the interface membrane 23. The distal wall 24 lies facing the interface membrane 23. In the example shown, the distal wall 24 is formed by a membrane, called the distal membrane 24, parallel to the interface membrane 23. The light source 10 is placed outside the internal space bounded by the cavity. The light beam propagates through the distal membrane 24, forming the distal wall of the cavity, then through the interface membrane 23, to the analyzed medium. The light beam 11 preferably propagates at a distance from the lateral wall 22 bounding the cavity 20, so as to avoid heating the latter, as otherwise a parasitic acoustic signal not specific to the analyte sought is formed. This is also the reason why the distal wall 24 of the cavity is preferably formed by a thin distal membrane, made of a material considered transparent in the emission spectral band. The distal membrane has a high transmittance in the emission spectral band, so as to maximize the amount of light propagating to the analyzed medium. The greater the amount of light reaching the medium, the higher the sensitivity of the device.

The volume of the cavity may be a few μL or tens of μL (microliters). In order to increase the amplitude of the pressure wave detected by the acoustic detector, it has been estimated that the volume of the cavity must preferably be less than 50 μL.

The light beam 11 reaching the medium 2 is gradually absorbed by the latter, and in particular by the analyte 4, according to the Beer-Lambert law of absorption. The rate of absorption increases as the concentration of analyte absorbing the light beam increases. Thus, the higher the concentration of analyte, the smaller the thickness of medium penetrated by the light beam. The optical energy absorbed by the analyte is transferred to the medium in the form of heat. The heat diffuses through the medium to the interface membrane. The interface membrane 23 then undergoes a periodic temperature variation, induced by the periodic heating of the medium, the periodic heating of the medium resulting from the periodic illumination by the light beam 11.

The periodic temperature variation of the interface membrane 23 generates, inside the cavity, a periodic pressure wave 12, the latter being detected by the acoustic detector 25. It is under the effect of the periodic heating of the membrane 23 that the pressure wave is formed. The photoacoustic effect is indirect, since it is generated at the interface membrane, under the effect of the conduction of heat through the medium. The period of the pressure wave formed in the cavity corresponds to the period of the incident light beam 11.

In order for the transduction of the temperature variation of the medium into the amplitude variation of the pressure wave to be optimal, it is preferable for the thermal conductivity of the material forming the interface membrane to be high, for example higher than 0.5 W·m⁻¹·K⁻¹ or 1 W·m⁻¹·K⁻¹ or 5 W·m⁻¹·K⁻¹ or 10 W·m⁻¹·K⁻¹. It is also preferable for the material forming the membrane to have a low density. It is preferable to avoid materials such as plastics or textiles. The specific heat capacity of the material forming the interface membrane is preferably low. Lastly, the interface membrane is preferably thin, as described with reference to FIG. 2.

Another function of the interface membrane is to prevent some of the analyzed medium from penetrating into the cavity. Thus, the interface membrane forms a fluid barrier, in particular with respect to liquids or gels likely be present in the analyzed medium. This prevents contamination of the interior of the cavity. This also makes it possible to control the volume of gas inside the cavity. Indeed, a variation in the internal volume of the cavity may lead to bias in the interpretation of the measurement taken by the acoustic detector. This bias may affect the relationship between the amplitude of the photoacoustic wave and the concentration of the analyte. Thus, by blocking passage of the liquid or gel into the interior of the cavity, the interface membrane allows the response function of the device to be stabilized.

FIG. 2 schematically shows one example of the interface membrane 23. The interface membrane 23 extends between an external surface 23ₑ, which is intended to make contact with the analyzed medium, and an internal surface 23ᵢ, which makes contact with the gas present in the cavity. Between the external surface and the internal surface, the membrane has a small thickness ε, preferably one less than 1 mm. Advantageously, the thickness ε of the interface membrane 23 is comprised between 25 μm and 100 μm. The thicker the membrane, the less heat conducted to the cavity.

The interface membrane comprises an intersecting segment 23ᵢₙₜ, corresponding to the portion of the interface membrane passed through by the light beam 11. At least in the intersecting segment 23ᵢₙₜ, the interface membrane is formed from a material having a high transmittance in the spectral band Δλ of the emitted beam 11. By high transmittance, what is meant is a transmittance that is preferably higher than 0.4 or even and preferably higher than 0.8, and for example of the order of 0.9 or more. By transmittance, what is meant is a fraction of the light intensity transmitted by the interface membrane 23. The interface membrane may be partially or entirely formed from Si, or some other material transparent in the infrared, for example porous Si, Ge, AlN, ZnSe, BaF2, CaF2, KBr, ZnS, or sapphire. The same goes for the distal membrane 24 described above. A high transmittance allows the amount of light reaching the medium to be maximized.

Transmittance may be increased, to reach values close to 1, by applying an anti-reflection coating, in particular to the internal surface 23ᵢ and preferably to the internal surface 23ᵢ and to the external surface 23ₑ. The anti-reflection coating may take the form of an unapertured “quarter-wave” layer deposited in the form of a thin layer, at least in the intersecting segment 23ᵢₙₜ. Alternatively, the anti-reflection treatment may consist of micro-structuring of the internal surface 23ᵢ, and optionally of the external surface 23ₑ, at least in the intersecting segment 23ᵢₙₜ through which the light beam propagates. The micro-structuring may for example form a diffraction grating, allowing transmission of light in the emission spectral band to be optimized. One example of micro-structuring is described in Douglas S. Hobbs, Brue D. Macleod and Juanita R. Riccobono “Update on the development of high performance anti-reflecting surface relief micro-structures”, Proc. SPIE 6545.

The interface membrane may be monolithic, being formed from a single material, or composite. When the interface membrane is composite, it may comprise a material considered sufficiently transparent in the emission spectral band, in the intersecting segment 23ᵢₙₜ, and another material outside the intersecting segment, for example a very good thermal conductor, such as a metal like copper or aluminum.

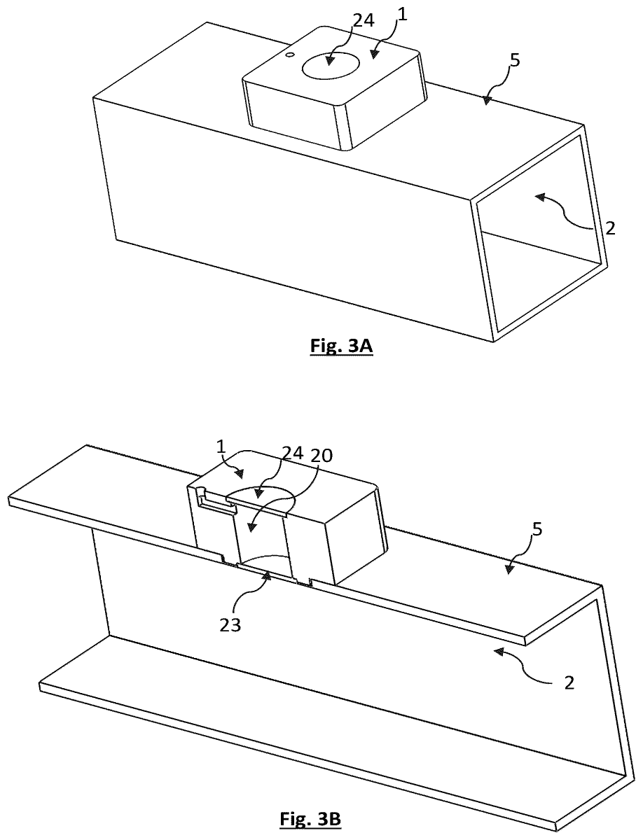

FIG. 3A schematically shows one possible way in which a device 1 such as described above may be applied to a duct 5 through which a liquid 2 flows. The interface membrane 23 is placed in an aperture in the duct, at the interface between the liquid and the cavity. FIG. 3B is a cutaway view of FIG. 3A, showing a median plane.

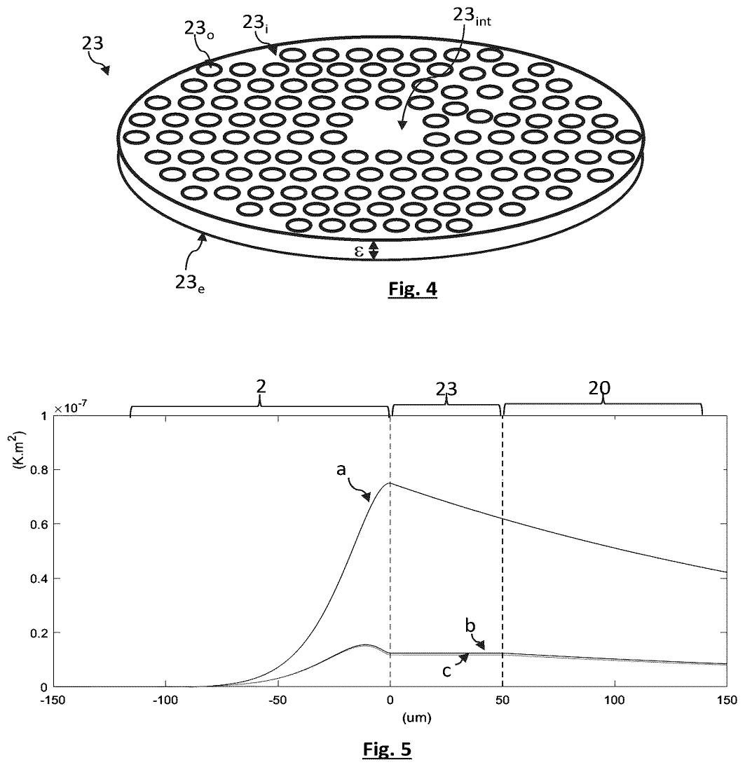

In the example shown in FIG. 2, the interface membrane 23 is unapertured. According to one possibility, shown in FIG. 4, the interface membrane 23 comprises through-apertures 23ₒ, extending right through the membrane. The radius of the through-apertures is preferably comprised between 5 μm and 50 μm, and preferably between 20 and 30 μm. The apertures are advantageously functionalized, for example with a hydrophobic treatment, to prevent passage of liquid through the membrane. Such a configuration makes it possible to take advantage of a photoacoustic wave generated, in each aperture, at the interface between the medium and the air of the cavity. According to this possibility, the intersecting segment 23ᵢₙₜ of the membrane 23 is preferably unapertured, so that the through-apertures 23ₒ do not disrupt propagation of the light beam through the interface membrane.

In the configuration of FIG. 4, it is advantageous, but not essential, for the membrane to be a good thermal conductor. Thus, in this embodiment, the membrane may be a polymer containing a multitude of apertures.

The amplitude of the thermal oscillations as a function of position, along the transverse axis Z, in the medium 2 and in the cavity 20, under the effect of periodic illumination, has been simulated. FIG. 5 shows the amplitude of the thermal oscillations (y-axis—unit K·m²), as a function of the z-position along an axis parallel to the axis Z, the axis being centered with respect to the cavity and to the membrane. This axis has been represented by a dash-dotted line in FIG. 1. Positions z<0 correspond to the analyzed medium 2. Positions z>0 correspond to the device. The coordinate z=0 corresponds to the medium/device interface. First, a device without a membrane was considered, the corresponding results being plotted in curve a). Next, a membrane 23 of 50 μm thickness was considered. The modeling parameters were as follows:
sample: water at a temperature of 25° C., thickness larger than 1 mm;
illumination beam: laser source of 10 mW power modulated at a frequency of 100 Hz—wavenumber: 1035 cm⁻¹.
membrane constitution: Si (curve b) or Ge (curve c), without anti-reflection treatment.

In FIG. 5, the curves corresponding to the Si or Ge membrane are almost coincident.

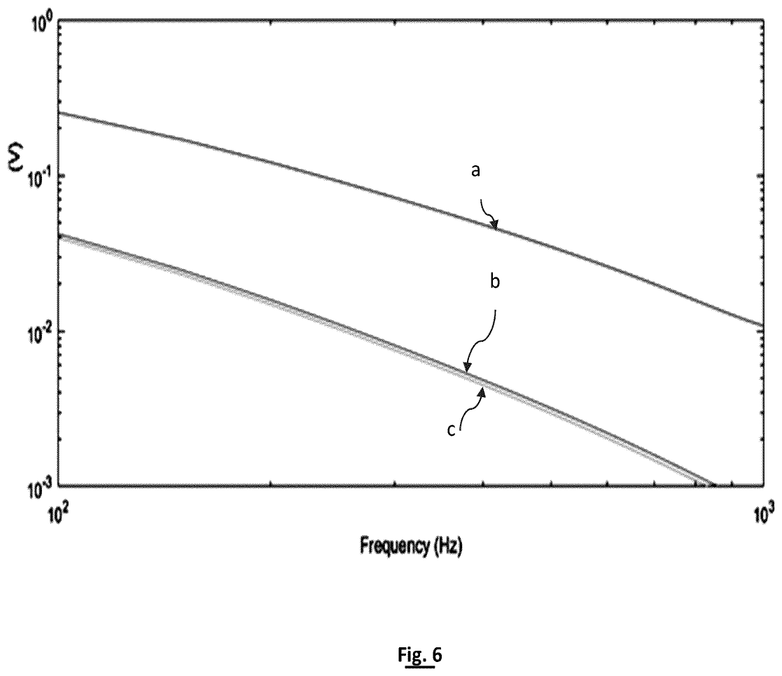

FIG. 6 shows the variation, as a function of modulation frequency (x-axis—Hz), in the amplitude of the signal measured by an acoustic detector (y-axis—unit V), which is representative of the photoacoustic wave.

FIG. 5 shows that the photothermal effect, i.e. heating of the analyzed medium by the laser beam, occurs to a depth smaller than 100 μm from the interface membrane. Moreover, temperature stability is observed in the thickness of the membrane. This is due to the large thermal diffusion length of Si and Ge. This confirms that these materials are well suited to form the interface membrane. Moreover, the very similar values obtained for Si and Ge are due to the good optical-transmission properties, in the infrared, of these two materials.

FIG. 6 shows that at a modulation frequency of 100 Hz, the amplitude of the photoacoustic wave is attenuated by a factor approximately equal to 10 in the presence of the membrane (curves b and c in FIG. 6) compared to the configuration with no membrane (curve a in FIG. 6). The attenuation induced by the interface membrane is attributed to effects of reflection of the light beam 11 as it propagates through the interface membrane, and in particular at the internal surface 23ᵢ and external surface 23ₑ. These unwanted reflections may be avoided by applying an anti-reflection treatment to these two surfaces. The inventors estimate that use of an anti-reflection treatment on the internal surface and external surface is liable to increase the amplitude of the photoacoustic pressure wave by a factor approximately equal to 4.

It will be noted that the amplitude of the photoacoustic wave decreases as modulation frequency increases, in all three configurations modeled: no membrane, Si membrane and Ge membrane. This is due to the fact that thermal diffusion length in the membrane, and in air, decreases as frequency increases. It may be seen in FIG. 6 that the difference between curve a (no membrane) and curves b) and c) (Si or Ge membrane) increases as modulation frequency increases. It is therefore preferable for the modulation frequency to be relatively low, for example lower than 100 Hz or a few hundred Hz. The membrane acts as a low-pass filter to the photoacoustic wave.

The interface membrane 23 may be manufactured independently of the cavity, and attached to the latter. It may be removable.

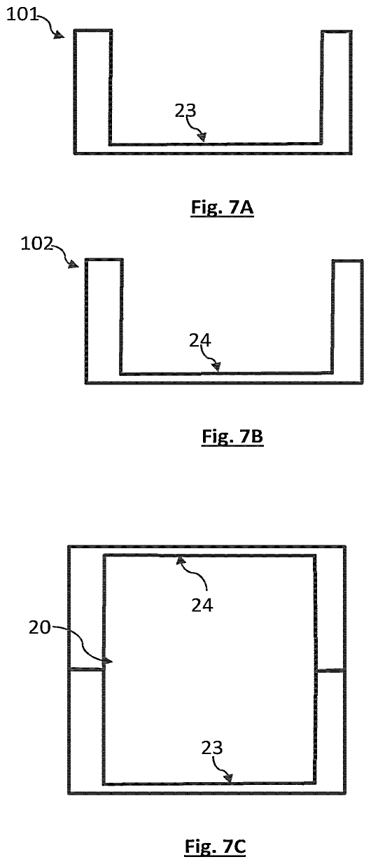

According to one possibility, the cavity is obtained by assembling two substrates. A first substrate 101 is etched, so as to form one portion of the cavity, and the interface membrane 23: see FIG. 7A. At the level of the interface membrane 23, the substrate is thinned so as to obtain a membrane of desired thickness. A second substrate 102 is etched, so as to form a complementary portion of the cavity, and optionally the distal membrane 24. See FIG. 7B. The substrates 101 and 102 are sealed against each other, so as to form the cavity bounded on the one hand by the interface membrane 23 and on the other hand by the distal membrane 24: see FIG. 7C.

The device may comprise a biocompatible material, configured to extend over the interface between the analyzed medium and the membrane. The biocompatible material is chosen so that long-term biocompatibility is assured, and so that optimal transmission of the heat transferred by the analyzed medium to the membrane is allowed. The biocompatible material may be a metal, for example aluminum or copper. Its thickness may then be smaller than or of the order of 1 μm. The biocompatible material may be a plastic, in which case the thickness is a few tens or hundreds of microns.

The invention will potentially be employed to measure concentrations of molecules of interest in a medium, in particular a liquid medium or a gel. The applications may concern the health field, but also industrial fields such as the food industry or the pharmaceutical or chemical industry.

1. A photoacoustic detecting device to be applied, via a contact face, against a medium to be analyzed, the device comprising:
a hollow cavity that is filled with a gas, and that opens onto the contact face;
a light source that is configured to emit, when it is activated, an incident light beam, in an emission spectral band, through the cavity, to the contact face, the incident light beam being pulsed or amplitude-modulated; and
an acoustic detector connected to the cavity;
wherein, under an effect of illumination of the medium by the incident light beam, the acoustic detector detects an acoustic wave produced by heating of the medium;
the device further comprises an interface membrane, forming the contact face, the interface membrane being configured to:
form an interface between the gas, filling the cavity, and the medium to be analyzed; and
block passage of the medium to be analyzed into the cavity; and
the acoustic detector is configured to detect an acoustic pressure wave, generated inside the cavity, under the effect of a variation in the temperature of the interface membrane, the temperature variation of the interface membrane being induced by the heating of the medium resulting from the illumination of the medium.

2. The device as claimed in claim 1, wherein the interface membrane is unapertured.

3. The device as claimed in claim 1, wherein the interface membrane comprises through-apertures of radius less than 50 μm or than 30 μm.

4. The device as claimed in claim 3, wherein the interface membrane comprises a hydrophobic coating in the through-apertures.

5. The device as claimed in claim 1, wherein:
the light source is arranged such that, when it is activated, the incident light beam passes through the interface membrane before reaching the medium to be analyzed;
the interface membrane comprises an intersecting segment, corresponding to a portion of the membrane passed through by the light beam; and
at least in the intersecting segment, the interface membrane is made of a transmissive material having a transmittance higher than 0.4 in the emission spectral band.

6. The device as claimed in claim 5, wherein the transmissive material is at least one material selected from: Si, Ge, AlN, ZnSe, BaF₂, CaF₂, KBr, ZnS, and sapphire.

7. The device as claimed in claim 1, wherein the interface membrane is removable.

8. The device as claimed in claim 1, wherein:
the interface membrane extends between an internal surface, making contact with the gas filling the cavity, and an external surface, intended to be applied against the medium to be analyzed;
the internal surface of the interface membrane comprises an anti-reflection coating or micro-structuring configured to minimize reflection of the light beam; and
the external surface of the interface membrane comprises an anti-reflection coating or micro-structuring configured to minimize reflection of the light beam.

9. The device as claimed in claim 1, wherein the thickness of the interface membrane is comprised between 20 μm and 1 mm.

10. The device as claimed in claim 1, wherein:
the cavity is bounded by a distal membrane, the distal membrane lying opposite the interface membrane, so that the cavity extends between the distal membrane and the interface membrane; and
the light source is arranged in such a way that, when it is activated, the incident light beam passes through the distal membrane before reaching the interface membrane.

11. The device as claimed in claim 1, wherein:
the cavity is bounded by a distal wall and a lateral wall, the lateral wall extending between the distal wall and the interface membrane; and
the interface membrane extends between opposite edges of the sidewall.

12. The device as claimed in claim 1, wherein the volume of the cavity is less than 50 μL.

13. The device as claimed in claim 1, wherein the acoustic detector is connected to the cavity by an acoustic channel.

14. The device as claimed in claim 1, wherein the interface membrane is formed from a material the thermal conductivity of which is higher than 0.5 W·m⁻¹·K⁻¹.

15. A method for detecting an analyte in a medium, the analyte absorbing light at an absorption wavelength, the method comprising:
applying the device as claimed in claim 1 against the medium, so that the interface membrane makes contact with the medium;
activating the light source, the emission spectral band containing the absorption wavelength of the analyte; and
detecting a photoacoustic pressure wave by means of the acoustic detector and estimating an amount of analyte depending on the detected photoacoustic pressure wave.

16. The method as claimed in claim 15, wherein the medium is liquid or is a gel.

17. The method as claimed in claim 15, wherein the light source is pulsed or amplitude-modulated, with a pulse frequency or modulation frequency less than 500 Hz.
